(19) United States
(12) Patent Application Publication
(10) Pub. No.: US 2011/0014282 A1
(43) Pub. Date: Jan. 20, 2011

(54) PHARMACEUTICAL COMPOSITION FOR POORLY SOLUBLE DRUGS

(76) Inventor: Teófilo Cardoso de Vasconcelos, Perafita (PT)

Correspondence Address: FOLEY & LARDNER LLP, 111 HUNTINGTON AVENUE, 26TH FLOOR, BOSTON, MA, 02199-7610, US

(21) Appl. No.: 12/920,025

(22) PCT Filed: Feb. 27, 2009
(86) PCT No.: PCT/PT2009/000009

§ 371 Date: Aug. 27, 2010

Related U.S. Application Data
(60) Provisional application No. 61/032,190, filed on Feb. 28, 2008.

(51) Int. Cl. A61K 9/48; A61K 31/4439; A61P 29/00; A61P 43/00; A61P 25/24; A61P 25/00; A61P 21/02; A61P 25/08; A61P 25/16; A61P 1/08; A61P 25/22; A61P 25/18; A61P 25/28; A61P 9/00; A61P 9/06; A61P 9/12; A61P 9/08; A61P 3/00

(52) U.S. Cl. 424/452; 514/340

(57) ABSTRACT

A pharmaceutical composition containing a solid dispersion of a poorly soluble active pharmaceutical ingredient, an amorphous carrier and a surfactant.

The present invention relates to a pharmaceutical composition with improved dissolution properties. More specifically the invention relates to fast release pharmaceutical compositions containing a solid dispersion of a poorly soluble active pharmaceutical ingredient, an amorphous carrier and a surfactant.

### BACKGROUND TO THE INVENTION

Although pharmaceuticals may be administered in a variety of ways, ease of administration means that oral drug delivery is the preferred administration route. Solid oral dosage forms are particularly preferred since these offer greater drug stability, more accurate dosing and easier production. However, for the treatment to be effective the oral dosage form must yield an effective and reproducible in vivo plasma concentration following administration. The oral dosage form must readily release the drug for its absorption.

The majority of new pharmaceuticals are poorly water soluble and are therefore not well-absorbed after oral administration. Moreover, absorption of most drugs takes place in the upper small intestine and is greatly reduced after the ileum, meaning that the absorption window is small. One of the current challenges in the pharmaceutical industry is the development of strategies that improve drug bioavailability, for example through development of fast release formulations which ensure that the drug is released in the short timeframe required for its uptake, or by improving drug solubility.

One such strategy has been the development of solid dispersions. Solid dispersions can be described as molecular mixtures of the active pharmaceutical ingredient (API) in hydrophilic carriers, wherein molecules of the carrier interact with API molecules such that the latter are distributed amongst the carrier molecules. In a solid dispersion, the API is in a supersaturated state due to forced solubilisation in the carrier.

Initially first generation solid dispersions used crystalline carriers. In these dispersions the API molecules were incorporated in the crystal lattice of the carrier, either by taking the place of some of the carrier molecules in the lattice or by insertion amongst the carrier molecules without affecting the lattice structure. However, later developments used amorphous carriers, which, due to lower thermodynamic stability, were able to release the drug more rapidly from the dispersion.

Although such solid dispersions generally result in a greatly improved solubility of the API, problems still remained. One such problem is the stability of the API since, during processing or storage, the amorphous state may undergo recrystallisation. Many of the polymers used in solid dispersions absorb water, which may result in phase separation, crystal growth or conversion to a more stable crystalline state. All of these result in decreased solubility and reduced dissolution rate.

Third generation solid dispersions involve the dispersal of the API in a mixture of an amorphous carrier and a surfactant. These dispersions are aimed at maximising bioavailability for poorly soluble drugs as well as improving drug stability by overcoming the problem of drug recrystallisation. In addition to improving API dissolution, the inclusion of the surfactant was postulated to prevent precipitation and/or protect a fine crystalline precipitate from agglomeration into much larger hydrophobic particles. (Tanaka et al (2005), Development of novel sustained-release system, disintegration-controlled matrix tablet with solid dispersion granules of nilvadipine. Journal of Controlled Release 108 (2-3), 386-395).

The inventor has discovered that the inclusion of a much lower level of surfactant in the solid dispersion results in a surprisingly large increase in solubility for very insoluble drugs. Moreover, the inventor has discovered that the solid dispersion resulted in a very rapid release of the drug. Indeed, even when compressed, the use of a disintegrant in the solid dispersion was not required and very good dissolution resulted. The solid dispersion formulation also remained physically stable over a long period of time without significant drug recrystallisation.

### DESCRIPTION OF THE INVENTION

According to one aspect of the present invention, there is provided a solid oral dosage form of a poorly soluble active pharmaceutical ingredient (API), the oral dosage form comprising a solid dispersion of a poorly soluble API, an amorphous carrier and a surfactant, wherein the amount of surfactant is from 0.5 to 30% of the total weight of the solid dispersion and at least part of the API is in an amorphous form.

Preferably the dosage form is a fast-release dosage form. A fast release composition or dosage form is particularly one which dissolves rapidly, that is, one in which more than 85% of the labelled amount of drug substance dissolves within 60 minutes, preferably in less than 30 minutes in a volume of less than 1000 ml of either water or one of the three USP buffers listed below, measured using USP 31, apparatus I or II (See USP 31 chapter <711>-Dissolution, pages 267-274, 2008, Rockville).

The dosage form may also be a sustained release dosage form in which case the invention provides a dosage form in which more of the poorly soluble API is released when compared to the prior art. In this case, when such a dosage form is placed in a volume of less than 1000 ml of water, more than 85% of the API dissolves in less than 12 hours, for example less than 10 hours, less than 8 hours or less than 6 hours.

USP Buffers:

Hydrochloric Acid Buffer pH 1.2 (USP 31, NF28, 2008, Rockville)

Place 50 mL of the potassium chloride solution 0.2M in a 200-mL volumetric flask, add 85 ml of the hydrochloric acid solution 0.2 M, then add water to volume

Acetate Buffer, pH 4.5 (USP 31, NF28, 2008, Rockville)

Place 2.99 g of sodium acetate NaC₂H₃O₂.3H₂O in a 1000-mL volumetric flask, add 14.0 ml of the acetic acid solution 2 N, then add water to volume, and mix.

Phosphate Buffer, pH 6.9 (USP 31, NF28, 2008, Rockville)

Place 50 mL of the monobasic potassium phosphate solution 0.2 M in a 200-mL volumetric flask, add 25.8 ml of the sodium hydroxide solution 0.2 M, then add water to volume.

The oral dosage form may be a capsule dosage form wherein granules of the solid dispersion are contained within an outer casing of a pharmaceutically acceptable material. Suitable materials for the outer casing will be well-known to those skilled in the field but include casings of gelatine or HPMC. Additional substances such as excipients may also be contained within the outer casing.

Alternatively, the oral dosage form is a compressed dosage form, such as a tablet, wherein granules of the solid dispersion are compressed into a tablet matrix.

Preferably the compressed dosage form has a resistance to a crushing force of from 0.1N to 300N, more preferably still of from 20N to 200N.

Preferably the solid dispersion does not include a superdisintegrant.

Preferably the solid oral dosage form does not contain a superdisintegrant. However, for a compressed dosage form, whilst the solid dispersion granules do not contain a superdisintegrant, the tablet matrix may include a superdisintegrant.

The term ‘superdisintegrant’ refers to a substance which highly promotes break down or disintegration of a composition, releasing its constituent particles. Superdisintegrants include carboxymethylcellulose calcium (ECG 505, Nymcel ZSC), carboxymethylcellulose sodium (Akucell, Aquasorb, Blanose, Finnfix, Nymcel Tylose CB), croscarmellose sodium (Ac-Di-SoI, Explocel, Nymcel ZSX, Pharmacel XL, Primellose, Solutab, Vivasol), and sodium starch glycolate (Explotab, Primojel, Vivastar P).

Preferably at least 30% of the API is present in an amorphous form. More preferably at least 50% of the API is in an amorphous form. More preferably still at least 75% of the API is in an amorphous form. Most preferably at least 90% of the API is in an amorphous form.

Surfactant

Preferably the amount of surfactant in the solid dispersion is from 0.5% to less than 30%, more preferably less than 10%, still more preferably from 2% to 24%, yet more preferably from 2% to 16%, more preferably still from 2% to 10%, and most preferably from 4 to 8% of the total weight of the solid dispersion.

Suitable surfactants include inulin (inutec), mono-, di- and triglycerides of behenic acid (compritol), glycerol and PEG1500 esters of long fatty acids (gelucire), sodium docusate, self emulsifying glyceryl monooleate (tegin), cetrimide, polyoxyethylene alkyl ethers (brij), polyoxyethylene castor oil derivates(simusol), polyoxyethylene stearates (Hadag, Kessco), sorbitan esters (span), poloxamer (pluronics), sodium lauryl sulphate and polysorbates.

Preferably the surfactant is a non-ionic surfactant.

Preferably the surfactant is a polysorbate, more preferably polysorbate 80. The surfactant polysorbate is also known by its commercial name Tween. Thus preferably the surfactant is Tween 80, or T80.

Alternatively the surfactant is sodium lauryl sulphate.

The term ‘wetting agent’ may be used to denote the term ‘surfactant’.

Active Pharmaceutical Ingredient (API)

APIs typically suited to the formulation of the invention are those classed in Biopharmaceutics Classification System (BCS) class II. A BCS class II (sometimes referred to as Case II) drug is characterized by being poorly soluble and having high permeability. (Amidon, G. L.; Lennernäs, H.; Shah, V. P.; Crison, J. R., 1995, A theoretical basis for a biopharmaceutic drug classification: The correlation of in vitro drug product dissolution and in vivo bioavailability, Pharmaceutical research, 12, 413-420).

A poorly soluble API is defined as an API that, in its highest dosage administrable to humans, is not soluble in 250 ml of water-based buffers with a pH between 1-7.5. (Rinaki, E.; Valsami, G.; Macheras, P, 2003, Quantitative Biopharmaceutics Classification System: the central role of dose/solubility ratio, Pharmaceutical Research, 20, 1917-1925; Amidon, G. L.; Lennernäs, H.; Shah, V. P.; Crison, J. R., 1995, A theoretical basis for a biopharmaceutic drug classification: The correlation of in vitro drug product dissolution and in vivo bioavailability, Pharmaceutical research, 12, 413-420;). Water-based buffers include water and those described earlier as USP Buffers. The highest dose of a drug administrable to humans can be, for example, less than 2 g, from 0.5 to 1 g, from 1 mg to 0.5 g, from 1 ug to 1 mg. Generally more than 0.1%, for example more than 1%, more than 10%, more than 20%, or more than 50% of a such a dose of a poorly soluble drug is not dissolved in 250 ml of water-based buffers with a pH between 1-7.5.

A drug is considered to have high permeability when the extent of its absorption in humans is determined to be ≧90% of an administered dose, based on mass-balance or in comparison to an intravenous reference dose. (Amidon, G. L.; Lennernäs, H.; Shah, V. P.; Crison, J. R., 1995, A theoretical basis for a biopharmaceutic drug classification: The correlation of in vitro drug product dissolution and in vivo bioavailability, Pharmaceutical research, 12, 413-420)

### Typical BCS Class II Drugs Include:

Anti-infectious drugs such as Albendazole, Acyclovir, Azithromycin, Cefdinir, Cefuroxime axetil, Chloroquine, Clarithromycin, Clofazimine, Diloxanide, Efavirenz, Fluconazole, Griseofulvin, Indinavir, Itraconazole, Ketoconalzole, Lopinavir, Mebendazole, Nelfinavir, Nevirapine, Niclosamide, Praziquantel, Pyrantel, Pyrimethamine, Quinine, and Ritonavir.
Antineoplasic drugs such as Bicalutamide, Cyproterone, Gefitinib, Imatinib, and Tamoxifen.
Biologic and Immunologic Agents such as Cyclosporine, Mycophenolate mofetil, Tacrolimus.
Cardiovascular Agents such as Acetazolamide, Atorvastatin, Benidipine, Candesartan cilexetil, Carvedilol, Cilostazol, Clopidogrel, Ethylicosapentate, Ezetimibe, Fenofibrate, Irbesartan, Manidipine, Nifedipine, Nilvadipine, Nisoldipine, Simvastatin, Spironolactone, Telmisartan, Ticlopidine, Valsartan, Verapamil, Warfarin.
Central Nervous System Agents such as Acetaminophen, Amisulpride, Aripiprazole, Carbamazepine, Celecoxib, Chlorpromazine, Clozapine, Diazepam, Diclofenac, Flurbiprofen, Haloperidol, Ibuprofen, Ketoprofen, Lamotrigine, Levodopa, Lorazepam, Meloxicam, Metaxalone, Methylphenidate, Metoclopramide, Nicergoline, Naproxen, Olanzapine, Oxcarbazepine, Phenyloin, Quetiapine, Risperidone, Rofecoxib, and Valproic acid.
Dermatological Agents such as Isotretinoin
Endocrine and Metabolic Agents such as Dexamethasone, Danazol, Epalrestat, Gliclazide, Glimepiride, Glipizide, Glyburide (glibenclamide), levothyroxine sodium, Medroxyprogesterone, Pioglitazone, and Raloxifene.
Gastrointestinal Agents such as Mosapride, Orlistat, Cisapride, Rebamipide, Sulfasalazine, Teprenone, and Ursodeoxycholic Acid.
Respiratory Agents such as Ebastine, Hydroxyzine, Loratadine, and Pranlukast

However, the skilled person will be well aware of other BCS class II drugs which can be used with the invention.

Preferred APIs suitable for use in the dosage form include drugs active on the central nervous system such as analgesics, antipyretics, headache drugs, antidepressants, muscular relaxants, antiepileptics, antiparkinsonian drugs, antiemetics, anxiolytics, drugs used in the treatment of bipolar disorder and Alzheimer disease, and antipsychotics.

Alternative preferred APIs suitable for use in the dosage form include cardiovascular drugs such as include cardiotonics, antiarrhythmics, sympathomimetics, anti-hypertensive, vasodilators and cholesterol lowering drugs.

Preferably the API is a COMT inhibitor, a FAAH inhibitor, a dopamine beta hydroxylase inhibitor, or a sodium channel antagonist.

In one embodiment the API is 5-[3-(2,5-dichloro-4,6-dimethyl-1-oxy-pyridine-3-yl)-[1,2,4]oxadiazol-5-yl]-3-nitrobenzene-1,2-diol.

In another embodiment the API is 5-[3-(2,5-dichloro-4,6-dimethylpyridine-3-yl)-[1,2,4]oxadiazol-5-yl]-3-nitrobenzene-1,2-diol.

Alternative APIs include 5-[(1E)-2-(4-hydroxyphenyl)ethenyl]-1,3-benzenediol and 1-(3,4-dihydroxy-5-nitrophenyl)-2-phenyl-ethanone.

Carrier

Preferably the amorphous carrier is a polymer such as a cellulose derivative, starch derivative, polyethyleneglycol (PEG), polymethylacrylate, carbomer, polyvinyl acetate, povidone, crospovidone, D-alpha-tocopheryl poly(ethylene glycol) 1000 succinate (TPGS 1000) or vinylpyrrolidone/vinylacetate copolymer (copovidone, PVP VA64).

Suitable cellulose derivatives include hydroxylpropylmethylcellulose, ethylcellulose, methylcellulose, hydroxypropylcellulose and hypromellose acetate succinate (HPMC-AS).

Suitable starch derivatives include cyclodextrins.

Preferably the amorphous carrier is a polyethylene glycol having a molecular mass from 3000 to 20 000 g/mol, even more preferably from 4000 to 10 000 g/mol. Most preferably PEG has a molecular mass of 6000 g/mol.

Preferably the API and amorphous carrier are present in a API/carrier ratio of 1:from 0.5 to 1.5, most preferably 1:1.

Preferably, the API/amorphous carrier/surfactant ratio is from 25 to 65:from 25 to 65:from 0.5 to 30.

Preferably, the API/amorphous carrier/surfactant ratio is from 35 to 49.7:from 35 to 49.7:from 0.5 to 24.

More preferably, the API/amorphous carrier/surfactant ratio is from 45 to 49:from 45 to 49:from 2 to 10.

In a most preferred embodiment, the API/amorphous carrier/surfactant ratio is from 46 to 48:from 46 to 48:from 4 to 8.

The dosage form of the invention may comprise a further substance. The further substance may be any excipient.

Preferably the excipient is a filler and/or a lubricant. Suitable fillers and lubricants are described below.

Suitable fillers include calcium carbonate (Barcroft, Cal-Carb, CalciPure, Destab, MagGran, Millicarb, Pharma-Carb, Precarb, Sturcal, Vivapres Ca), calcium phosphate, dibasic anhydrous (A-TAB, Di-Cafos A-N, Emcompress Anhydrous, Fujicalin), calcium phosphate, dibasic dihydrate (Cafos, Calipharm, Calstar, Di-Cafos, Emcompress), calcium phosphate tribasic (Tri-Cafos, TRI-CAL WG, TRI-TAB), calcium sulphate (Destab, Drierite, Snow White, Cal-Tab, Compactrol, USG Terra Alba), cellulose powdered (Arbocel, Elcema, Sanacel, Solka-Floc), silicified microcrystalline cellulose (ProSolv), cellulose acetate, compressible sugar (Di-Pac), confectioner's sugar, dextranes (Candex, Emdex), dextrin (Avedex, Caloreen, Crystal Gum, Primogran W), dextrose (Caridex, Dextrofin, Lycadex PF, Roferose, Tab fine D-100), fructose (Advantose, Fructamyl, Fructofin, Krystar), kaolinLion, Sim 90), lactitol (Finlac ACX, Finlac DC, Finlac MCX)5 lactose (Aero Flo 20, Aero Flo 65, Anhydrox, CapsuLac, Fast-Flo, FlowLac, GranuLac, InhaLac, Lactochem, Lactohale, Lactopress, Microfine, Microtose, Pharmatose, Prisma Lac, Respitose, SacheLac, SorboLac, Super-Tab, Tablettose, Wyndale, Zeparox), magnesium carbonate, magnesium oxide (MagGran MO), maltodextrin (C*Dry MD, Glucidex, Glucodry, Lycatab DSH, Maldex, Maltagran, Maltrin, Maltrin QD, Paselli MD 10 PH, Star-Dri), maltose (Advantose 100), mannitol (Mannogem, Pearlitol), microcrystalline cellulose (Avicel PH, Celex, Celphere, Ceolus KG, Emcocel, Ethispheres, Fibrocel, Pharmacel, Tabulose, Vivapur), polydextrose (Litesse), simethicone (Dow Corning Q7-2243 LVA, Cow Corning Q7-2587, Sentry Simethicone), sodium alginate (Kelcosol, Keltone, Protanal), sodium chloride (Alberger), sorbitol (Liponec 70-NC, Liponic 76-NC, Meritol, Neosorb, Sorbifin, Sorbitol Instant, Sorbogem), starch (Aytex P, Fluftex W, Instant Pure-Cote, Melojel, Meritena Paygel 55, Perfectamyl D6PH, Pure-Bind, Pure-Cote, Pure-Dent, Pure-Gel, Pure-Set, Purity 21, Purity 826, Tablet White), pregelatinized starch (Instastarch, Lycatab C, Lycatab PGS, Merigel, National 78-1551, Pharma-Gel, Prejel, Sepistab ST 200, Spress B820, Starch 1500 G, Tablitz, Unipure LD, Unipure WG220), sucrose, trehalose and xylitol (Klinit, Xylifm, Xylitab, Xylisorb, Xylitolo).

The term ‘filler’ is sometimes used interchangeably with the term ‘diluent’. However, the term ‘filler’ is generally used for solid formulations whereas the term ‘diluent’ is used in liquid formulations.

Suitable lubricants include calcium stearate (HyQual), glycerine monostearate (Capmul GMS-50, Cutina GMS, Imwitor 191 and 900, Kessco GMS5 Lipo GMS 410, 450 and 600, Myvaplex 600P, Myvatex, Protachem GMS-450, Rita GMS, Stepan GMS, Tegin, Tegin 503 and 515, Tegin 4100, Tegin M, Unimate GMS), glyceryl behenate (Compritol 888 ATO), glyceryl palmitostearate Precirol ATO 5), hydrogenated castor oil (Castorwax, Castorwax MP 70, Castorwax MP 80, Croduret, Cutina HR, Fancol, Simulsol 1293), hydrogenated vegetable oil type I (Akofine, Lubritab, Sterotex, Dynasan P60, Softisan 154, Hydrocote, Lipovol HS-K, Sterotex HM), magnesium lauryl sulphate, magnesium stearate, medium-chain triglycerides (Captex 300, Captex 355, Crodamol GTC/C, Labrafac CC, Miglyol 810, Miglyol 812, Myritol, Neobee M5, Nesatol, Waglinol 3/9280), poloxamer (Lutrol, Monolan, Pluronic, Supronicm Synperonic), polyethylene glycol (Carbowax, Carbowax Sentry, Lipo, Lipoxol, Lutrol E, Pluriol E), sodium benzoate (Antimol), sodium chloride (Alberger), sodium lauryl sulphate (Elfan 240, Texapon K1 2P), sodium stearyl fumarate (Pruv), stearic acid (Crodacid E570, Emersol, Hystrene, Industrene, Kortacid 1895, Pristerene), talc (Altaic, Luzenac, Luzenac Pharma, Magsil Osmanthus, Magsil Star, Superiore), sucrose stearate (Surfhope SE Pharma D-1803 F) and zinc stearate (HyQual).

The invention will be further described with reference to the following examples which should not be intended to limit the scope of the claimed invention.

### DESCRIPTION OF DRAWINGS

FIG. 1 shows the effect of increasing drug content on the crystallinity of the solid dispersion of a poorly soluble BCS class II drug, ibuprofen (Drug A). Measurements were taken after 18 months of preparation of the solid dispersions. (SD=solid dispersion)

FIG. 2 shows the effects of varying drug content in solid dispersions which do not contain a surfactant on the poorly soluble BCS class II drug (Drug A). The solid dispersions contained only drug and carrier. The carrier used was PEG6000. (SD=solid dispersion; PM=physical mixture)

FIG. 3 shows the improvements in solubility achieved when a surfactant was included in the physical mixtures and solid dispersions with corresponding drug A:polymer carrier proportions. The drug and carrier were used in 1:1 ratio with the content of the surfactant increasing as shown in FIG. 3. (SD=solid dispersion; PM=physical mixture; T80=Tween 80)

FIG. 4 shows drug dissolution for tablets of the pure drug A; a solid dispersion of 1:1 drug A:carrier; the physical mixture of 1:1 drug A:carrier with surfactant; and a solid dispersion of 1:1 drug A:carrier with the surfactant, sodium lauryl sulphate (SLS).

FIG. 5 shows drug dissolution for tablets of the pure drug A; a solid dispersion of 1:1 drug A:carrier; physical mixtures of 1:1 drug A:carrier with two amounts of surfactant; and solid dispersions of 1:1 drug A:carrier with two amounts of surfactant. The surfactant used is Tween 80 (T80).

FIG. 6 shows drug dissolution for a solid dispersion of a poorly soluble BCS class II drug 1-(3,4-dihydroxy-5-nitrophenyl)-2-phenyl-ethanone (drug B) when formulated as a tablet of pure drug, of physical mixture of drug, carrier and surfactant, and of an equivalent solid dispersion. The surfactant used was Tween 80. The proportions used in the physical mixture and solid dispersion was drug:carrier:surfactant, 47:17:6.

FIG. 7 shows drug dissolution for a solid dispersion of a poorly soluble BCS class II drug 5-[(1E)-2-(4-hydroxyphenyl)ethenyl]-1,3-benzenediol (drug C) when formulated as a tablet of pure drug and of a solid dispersion of drug, carrier and surfactant. The surfactant used was Tween 80. The proportions used in the solid dispersion was drug:carrier:surfactant, 47:17:6.

### EXPERIMENTAL SECTION

### Materials and Methods

Solid dispersions were prepared by the common fusion method. Briefly, physical mixtures of drug, carrier and surfactant were heated at 90° C. i.e. above the melting point of the carrier. The drugs tested were: Ibuprofen (drug A), 1-(3,4-dihydroxy-5-nitrophenyl)-2-phenyl-ethanone (drug B), and 5-[(1E)-2-(4-hydroxyphenyl)ethenyl]-1,3-benzenediol (drug C).

The resulting melted products were stored at −5° C. for 24 hours in order to solidify completely. The samples were ground with a mortar and pestle and sieved with a 750 μm sieve.

Physical mixtures were prepared by mixing drug and surfactant with the carriers in a glass mortar and pestle.

Tablets of the solid dispersions, the physical mixtures and of the pure API were prepared by compression of a mass of physical mixture or solid dispersion or API containing 100 mg of drug in a hydraulic press with a 1 ton force for 5 seconds.

TABLE 5
Composition of the prepared formulations (percentage).

| Drug A | Drug B | Drug C | PEG 6000 | T80 | SLS |
|---|---|---|---|---|---|
| 50 | | | 50 | | |
| 49 | | | 49 | 2 | |
| 47 | | | 47 | 6 | |
| 47 | | | 47 | | 6 |
| | | | 47 | 6 | |
| | 47 | | 47 | 6 | |
| | | 47 | 47 | 6 | |
| | | | 47 | 6 | |
| | | | 47 | 6 | |

The following formulations were prepared:

### Solid Dispersion and Physical Mixture Composition

### DRUG—100 mg

### PEG 6000—100 mg

### Tween 80—13 mg

### Pure Drug Composition

### DRUG—100 mg

### Degree of Amorphization

Degree of amorphization was assessed after 1 year of storage under uncontrolled conditions of room temperature (15-25° C.) and humidity (approx. 65% humidity) using Differential Scanning Calorimetric data (DSC), and the following equation:

Percentage of crystallinity=(ΔHₛ/ΔH_{mdrug}×F)×100

where ΔHₛ is the melting enthalpy of the sample (J/g), ΔH_{mdrug} is the melting enthalpy of drug (J/g) and F is the weight fraction of drug in the sample. The percentage of crystallinity was used to compare the degree of amorphization induced by each carrier and manufacturing process.

DSC measurement was carried out in hermetically sealed aluminium pans using a DSC 141 (Setaram, France) calibrated with indium. Samples were heated on a single increasing run under a dry nitrogen gas purge between 30 and 150° C. at a rate of 10° C./min.

Solubility Studies

Solubility was determined in triplicate by using the shake flask method in USP KCl buffer pH 1.2. An excess amount of each product was added to each vial containing 15 ml of buffer; after closing, the mixture was vortexed for 3 min in order to facilitate appropriate mixing of samples within the buffer; mixtures were then stored for 3 h in a water bath at 37° C. and shaken every 5 minutes; mixtures were then filtrated through Millipore membrane filter (0.45 μm type HV) and the resulting solutions were assayed spectrophotometrically.

Dissolution Studies

Drug release was determined using USP apparatus 2 (rotating paddle method). This assay was performed in a dissolution tester VK 7020 (Vankel, USA), with on-line evaluation of the drug release with time by UV/VIS spectrophotometer, Cary 50 (Vankel, USA) through a peristaltic pump. The dissolution media consisting of 900 ml of water for drug C, USP HCl buffer (pH 1.20±0.05) for drug A, and USP phosphate buffer (pH 6.90±0.05) for drug B was maintained at 37.0±0.5° C. and agitated with a paddle stir rate of 100 rpm. Sample collection was performed through cannulas with polyethylene flow filter of 10 μm.

Tablets of raw drug, the physical mixtures or the solid dispersions containing 100 mg of drug were analyzed spectroscopically

Results

Stability

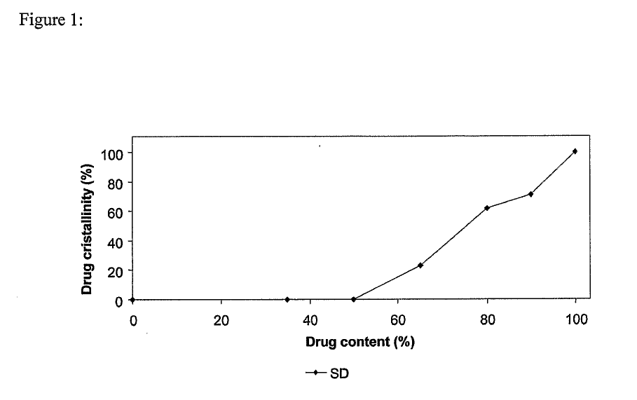

As can be seen in FIG. 1, for samples where the drug content was less than or equal to 50% of the solid dispersion, the solid dispersions are fully amorphous even after more than 12 months of storage. For all further solid dispersions the drug:carrier ratio was 1:1 to retain a fully amorphous state of the drug.

Solubility

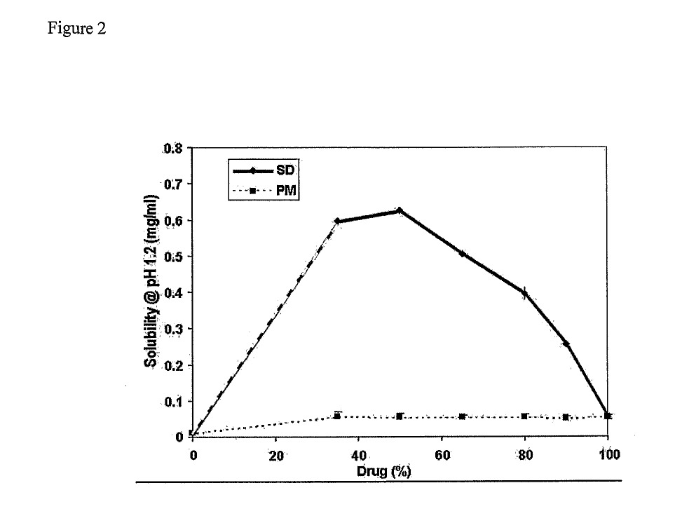

FIG. 2 represents a comparison between the solubility of a poorly soluble BCS Class II drug when in a physical mixture of a polymeric carrier and the drug and the same proportional mixture as a solid dispersion. As can be seen from FIG. 2, the solid dispersion provides an improvement in solubility when compared to its equivalent physical mixture for all samples tested.

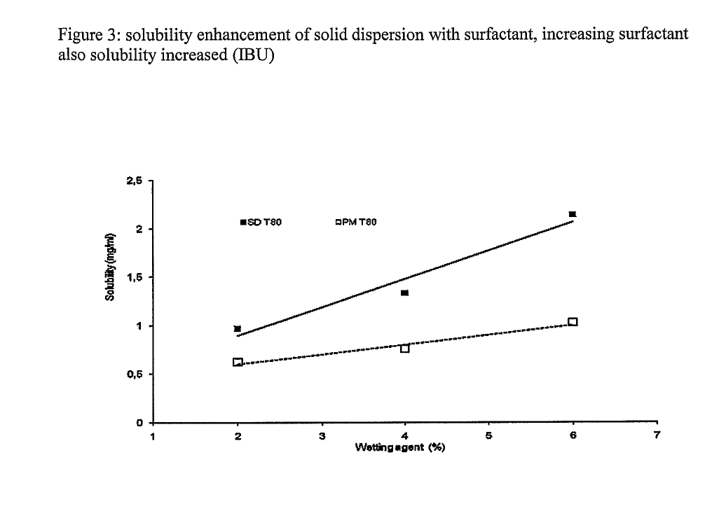

As can be seen from FIG. 3, the inclusion of a surfactant further improved solubility of the drug. Surprisingly, the results were greater when the surfactant was included in the solid dispersion when compared to inclusion in the equivalent physical mixture. This was particularly surprising given the, low levels of surfactant used.

Dissolution

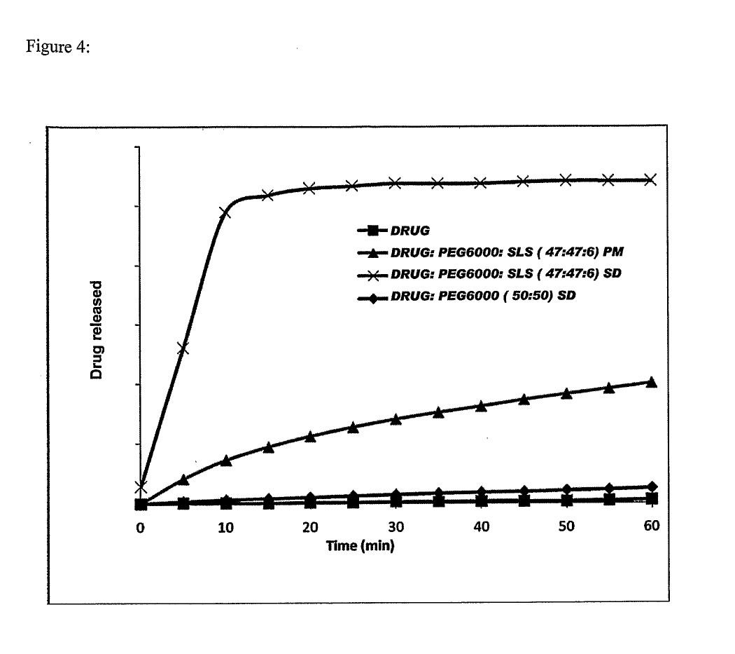

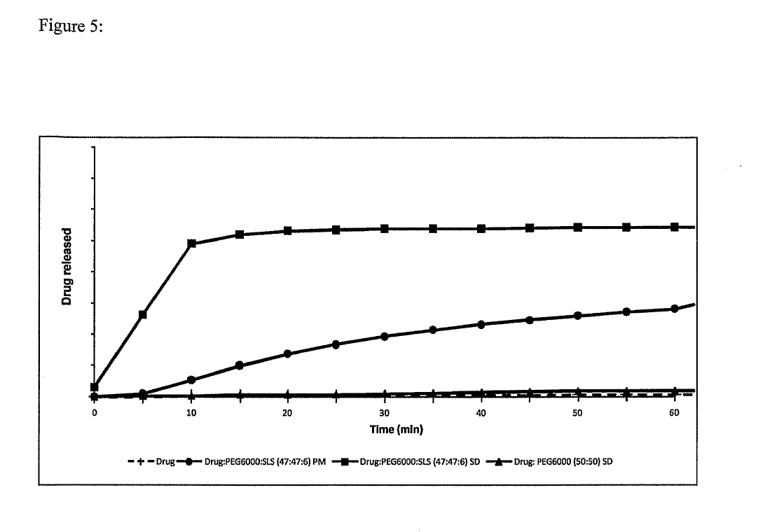

Surprisingly, given the improved solubility when formulating as a solid dispersion, (FIG. 2) the dissolution of the solid dispersion remained very poor (almost no change was seen compared with the pure drug): FIGS. 4 and 5. However, in addition to the improved solubility, an improvement in dissolution was seen when a surfactant was added to the physical mixture and to the solid dispersion. The effects were seen with two different surfactants.

However, the dissolution improvement was far greater than expected when the surfactant was added to the solid dispersion: as can be seen in FIGS. 4 and 5 not only is the drug released much faster but the drug release reaches a plateau, indicating that the majority of drug is released. As can be seen in FIG. 5, increasing the amount of surfactant increases the improvement in dissolution.

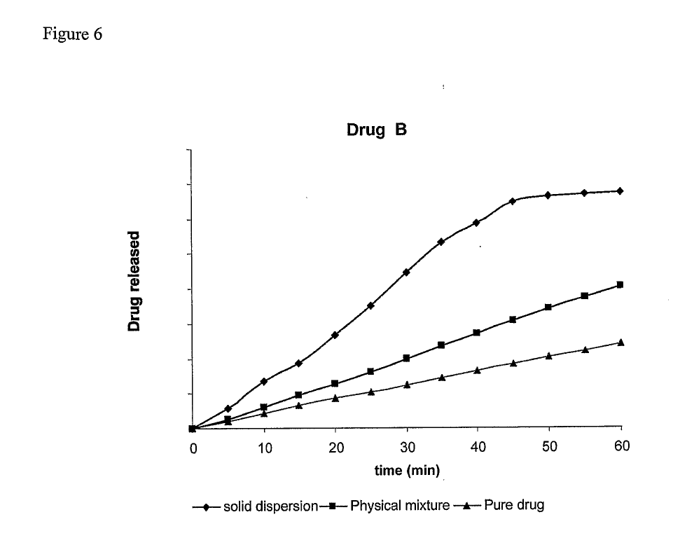

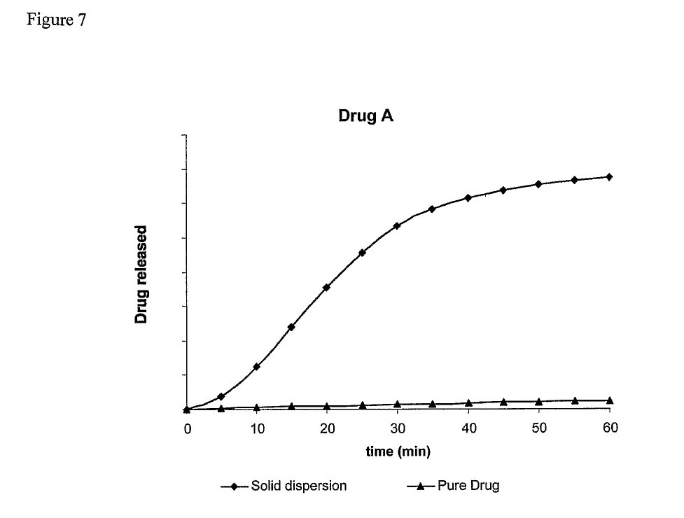

FIGS. 6 and 7 show that the effect is seen in a range of other BCS class II (poorly soluble) APIs.

The results shown above for a range of different poorly soluble drugs show that the inclusion of low levels of a surfactant in a solid dispersion improves the dissolution of the drug therefrom.

The improvements in dissolution result in greater release of the drug in a short space of time providing greater bioavailability, faster drug effect, reduced dosage levels, reduced side effects from reduced API and reduced surfactant levels and the reduction of food effect (effect of fed or fasted state of a patient on drug bioavailability). Tablet cost and size can also be reduced as a result of the invention.

Various modifications to the invention as described herein are within the scope of the appended claims.

1. A solid dosage form for release of a poorly soluble active pharmaceutical ingredient (API), the solid dosage form comprising a solid dispersion, the solid dispersion comprising a active pharmaceutical ingredient belonging to BCS Class II, an amorphous carrier and a surfactant, wherein the amount of surfactant is from 0.5 to 30% of the total weight of the solid dispersion, and wherein at least 30% of the active pharmaceutical ingredient is in an amorphous form.

2. A solid dosage form as claimed in claim 1 wherein the dosage form is for fast release of the API.

3. A solid dosage form as claimed in claim 1 or claim 2 wherein, when the dosage form is placed in a volume of less than 1000 ml of water, more than 85% of the API dissolves in less than 60 minutes.

4. A solid dosage form as claimed in claim 3 wherein, when the dosage form is placed in a volume of less than 1000 ml of water, more than 85% of the API dissolves in less than 30 minutes.

5. A solid dosage form as claimed in claim 1 wherein the dosage form is for sustained release of the API.

6. A solid dosage form as claimed in claim 5 wherein, when the dosage form is placed in a volume of less than 1000 ml of water, more than 85% of the API dissolves in less than 12 hours.

7. A solid dosage form as claimed in any preceding claim wherein the dosage form is a capsule formulation, the solid dispersion being contained within an outer casing of a pharmaceutically acceptable material.

8. A solid dosage form as claimed in any of claims 1 to 6 wherein the dosage form is a compressed dosage form.

9. A solid dosage form as claimed in claim 8 wherein the dosage form is a tablet.

10. A solid dosage form as claimed in claim 8 or claim 9 wherein the dosage form has a resistance to a crushing force of from 0.1 to 300 N.

11. A solid dosage form as claimed in claim 10 wherein the composition has a resistance to a crushing force of from 20 to 200 N.

12. A solid dosage form as claimed in any preceding claim wherein the solid dispersion does not contain a superdisintegrant.

13. A solid dosage form as claimed in any preceding claim wherein the dosage form does not contain a superdisintegrant.

14. A solid dosage form as claimed in any preceding claim wherein at least 50% of the active pharmaceutical ingredient is in an amorphous form.

15. A solid dosage form as claimed in claim 14 wherein at least 75% of the active pharmaceutical ingredient is in an amorphous form.

16. A solid dosage form as claimed in claim 15 wherein at least 90% of the active pharmaceutical ingredient is in an amorphous form.

17. A solid dosage form as claimed in any preceding claim wherein the amount of surfactant is from 2 to less than 24% of the total weight of the solid dispersion.

18. A solid dosage form as claimed in claim 17 wherein the amount of surfactant is from 2 to 16% of the total weight of the solid dispersion.

19. A solid dosage form as claimed in claim 18 wherein the amount of surfactant is from 2 to 10% of the total weight of the solid dispersion.

20. A solid dosage form as claimed in claim 19 wherein the amount of surfactant is from 4 to 8% of the total weight of the solid dispersion.

21. A solid dosage form as claimed in any preceding claim, wherein the surfactant is selected from inulin (inutec), mono-, di- and triglycerides of behenic acid (compritol 888 ATO), glycerol and PEG1500 esters of long fatty acids (gelucire), sodium docusate, self emulsifying glyceryl monooleate (tegin), cetrimide, polyoxyethylene alkyl ethers (brij), polyoxyethylene castor oil derivates (simusol), polyoxyethylene stearates (Hadag, Kessco), sorbitan esters (span), poloxamer (pluronics), sodium lauryl sulphate and polysorbates.

22. A solid dosage form as claimed in any of claims 1 to 20 wherein the surfactant is a non-ionic surfactant.

23. A solid dosage form as claimed in claim 22, wherein the surfactant is polysorbate 80.

24. A solid dosage form as claimed in any preceding claim wherein the active pharmaceutical ingredient is a drug which is active on the central nervous system.

25. A solid dosage form as claimed in claim 24 wherein the active pharmaceutical ingredient is selected from analgesics, antipyretics, headache drugs, antidepressants, muscular relaxants, antiepileptics, anticonvulsive drugs, antiparkinsonian drugs, antiemetics, anxiolytics, drugs used in the treatment of affective disorders such as bipolar disorder, antipsychotics and anti-Alzheimer drugs.

26. A solid dosage form as claimed in any of claims 1 to 23 wherein the active pharmaceutical ingredient is a COMT inhibitor, a FAAH inhibitor, a dopamine β hydroxylase inhibitor or a sodium channel antagonist.

27. A solid dosage form as claimed any of claims 1 to 23 wherein the active pharmaceutical ingredient is 5-[3-(2,5-dichloro-4,6-dimethyl-1-oxy-pyridine-3-yl)-[1,2,4]oxadiazol-5-yl]-3-nitrobenzene-1,2-diol.

28. A solid dosage form as claimed in any of claims 1 to 23 wherein the active pharmaceutical ingredient is 5-[3-(2,5-dichloro-4,6-dimethylpyridine-3-yl)-[1,2,4]oxadiazol-5-yl]-3-nitrobenzene-1,2-diol.

29. A solid dosage form as claimed in one of claims 1 to 23 wherein the active pharmaceutical ingredient is a cardiovascular active drug.

30. A solid dosage form as claimed in claim 29 wherein the active pharmaceutical ingredient is selected from cardiotonic drugs, antiarrhythmics, sympathomimetics, anti-hypertensives, vasodilators and cholesterol lowering drugs.

31. A solid dosage form as claimed in any preceding claim wherein the amorphous carrier is a polymer.

32. A solid dosage form as claimed in claim 31 wherein the polymer is selected from the group consisting of cellulose derivatives, starch derivatives, polyethyleneglycol, polymethylacrylate, carbomer, polyvinyl acetate, povidone, crospovidone, D-alpha-tocopheryl poly(ethylene glycol) 1000 succinate (TPGS 1000) and vinylpyrrolidone/vinylacetate copolymer (copovidone, PVP VA64).

33. A solid dosage form as claimed in claim 32 wherein the cellulose derivative is selected from the group consisting of hydroxylpropylmethylcellulose, ethylcellulose, methylcellulose, hydroxypropylcellulose and hypromellose acetate succinate.

34. A solid dosage form as claimed in claim 32 wherein the starch derivative is a cyclodextrin.

35. A solid dosage form as claimed in claim 32 wherein the polyethyleneglycol (PEG) is a PEG having a molecular mass from 3000 g/mol to 20000 g/mol.

36. A solid dosage form as claimed in claim 35 wherein the polyethyleneglycol is PEG6000.

37. A solid dosage form as claimed in any preceding claim wherein the active pharmaceutical ingredient and the amorphous carrier are present in a ratio of 1 part API to from 0.5 to 1.5 parts carrier.

38. A solid dosage form as claimed in claim 37 wherein the active pharmaceutical ingredient and the amorphous carrier are present in a ratio of 1:1.

39. A solid dosage form as claimed in any preceding claim wherein the ratio of the active pharmaceutical ingredient to amorphous carrier to surfactant is from 25 to 65:from 25 to 65:from 0.5 to 30.

40. A solid dosage form as claimed in claim 39 wherein the ratio of the active pharmaceutical ingredient to amorphous carrier to surfactant is from 35 to 49.7:from 35 to 49.7:from 0.6 to 24.

41. A solid dosage form as claimed in claim 40 wherein the ratio of the active pharmaceutical ingredient to amorphous carrier to surfactant is from 45 to 49:from 45 to 49:from 2 to 10.

42. A solid dosage form as claimed in claim 41 wherein the ratio of the active pharmaceutical ingredient to amorphous carrier to surfactant is from 46 to 48:from 46 to 48:from 4 to 8.

43. A solid dosage form as claimed in any preceding claim, wherein the composition comprises a filler.

44. A solid dosage form as claimed in any preceding claim, wherein the composition further comprises a lubricant.

45. A solid dosage form as claimed in any preceding claim, wherein the active pharmaceutical ingredient is not soluble in 250 ml of water-based buffers with a pH between 1-7.5.
